(19) United States
(12) Patent Application Publication
(10) Pub. No.: US 2015/0164398 A1
(43) Pub. Date: Jun. 18, 2015

(54) METHODS AND SYSTEMS FOR A SAMPLE COLLECTION DEVICE WITH A NOVELTY EXTERIOR

(71) Applicant: Theranos, Inc., Palo Alto, CA (US)

(72) Inventors: Pey-Jiun Ko, Palo Alto, CA (US); Elizabeth A. Holmes, Palo Alto, CA (US)

(21) Appl. No.: 14/538,753

(22) Filed: Nov. 11, 2014

Related U.S. Application Data
(60) Provisional application No. 61/902,777, filed on Nov. 11, 2013.

(51) Int. Cl. A61B 5/15; A61B 5/151; A61B 10/00

(52) U.S. Cl. CPC A61B 5/150839; A61B 5/150076; A61B 10/0045; A61B 5/151

(57) ABSTRACT

Methods for obtaining a sample from a subject include providing a sample collection device having a novelty exterior. The sample collection device can be used to collection liquid sample such as but not limited to blood, capillary blood, interstitial fluid, or other liquid sample. Samples may be provide by a small wound such as by a finger-stick and the sample may be analyzed in a short period of time, e.g., in less than five hours, or less than four hours.

### BACKGROUND

Clinical samples are useful in many situations, including being useful for monitoring the health of subjects, for diagnosing diseases or pathological conditions, and for monitoring the progress of therapeutic interventions, among various uses.

Obtaining clinical samples such as blood may be required for such uses; however, subjects may find providing such clinical samples to be painful, or difficult, or inconvenient. This can be particularly true for pediatric subjects who due to their age, smaller blood vessels, or other factor(s), can have difficulty with the sample collection process.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

At least some of disadvantages associated with the prior art are overcome by at least some or all of the embodiments described in this disclosure. Although the embodiments herein are typically described in the context of obtaining a blood sample, it should be understood that the embodiments herein are not limited to blood samples and can also be adapted to acquire other fluid(s) or bodily sample(s) for analysis.

In one embodiment described herein, a device is provided for collecting a bodily fluid sample. This embodiment may be useful for accurately collecting small volumes of bodily fluid sample that are often associated with non-venous blood draws. In one non-limiting example, the sample volume is about 1 mL or less. Optionally, the sample volume is about 900 uL or less. Optionally, the sample volume is about 800 uL or less. Optionally, the sample volume is about 700 uL or less. Optionally, the sample volume is about 600 uL or less. Optionally, the sample volume is about 500 uL or less. Optionally, the sample volume is about 400 uL or less. Optionally, the sample volume is about 300 uL or less. Optionally, the sample volume is about 200 uL or less. Optionally, the sample volume is about 100 uL or less. Optionally, the sample volume is about 90 uL or less. Optionally, the sample volume is about 80 uL or less. Optionally, the sample volume is about 70 uL or less. Optionally, the sample volume is about 60 uL or less. Optionally, the sample volume is about 50 uL or less.

In one non-limiting example, this device can be used to split the bodily fluid sample directly into two or more different portions that are then deposited into their respective containers. In one non-limiting example, the device comprises a first portion having at least two sample collection channels configured to draw the fluid sample into the sample collection channels via a first type of motive force, wherein one of the sample collection channels has an interior coating designed to mix with the fluid sample and another of the sample collection channels has another interior coating chemically different from said interior coating. The sample collection device includes a second portion comprising a plurality of sample containers for receiving the bodily fluid sample collected in the sample collection channels, the sample containers operably engagable to be in fluid communication with the collection channels, whereupon when fluid communication is established, the containers provide a second motive force different from the first motive force to move a majority of the bodily fluid sample from the channels into the containers. The containers may be arranged such that mixing of the fluid sample between the containers does not occur. Because this device may be used with non-venous draws, it may take a longer period of time to obtain a desired volume of sample and the early introduction of a material such as an anti-coagulant which may coat the channels, can prevent premature clogging of the channels during collection.

In another embodiment described herein, a device is provided for collecting a bodily fluid sample. The device comprises a first portion comprising a plurality of sample collection channels, wherein at least two of the channels are configured to simultaneously draw the fluid sample into each of the at least two sample collection channels via a first type of motive force. The device may also include a second portion comprising a plurality of sample containers for receiving the bodily fluid sample collected in the sample collection channels, wherein the sample containers have a first condition where the sample containers are not in fluid communication with the sample collection channels, and a second condition where the sample containers are operably engagable to be in fluid communication with the collection channels, whereupon when fluid communication is established, the containers provide a second motive force different from the first motive force to move bodily fluid sample from the channels into the containers.

In a still further embodiment described herein, a method is provided comprising metering a minimum amount of sample into at least two channels by using a sample collection device with at least two of the sample collection channels configured to simultaneously draw the fluid sample into each of the at least two sample collection channels via a first type of motive force. After a desired amount of sample fluid has been confirmed to be in the collection channels, fluid communication is established between the sample collection channels and the sample containers, whereupon the containers provide a second motive force different from the first motive force use to collect the samples to move bodily fluid sample from the channels into the containers. In some alternative embodiments, devices that use only a single channel to collect the body fluid or devices that have a plurality of channels but do not collect them simultaneously are not excluded. Optionally, the collection of sample fluid is performed without the use of a wicking material.

In one embodiment, there is a discrete amount of time between sample collection and introduction of the sample into a sample pre-processing device. In one non-limiting example, the process is a non-continuous process. The sample collection occurs in one processing station and then the sample is taken to a second station. This second station may be in the sample building. Optionally, the second station may be located at another location where the sample needs to be walked, driven, flown, conveyor-ed, placed in a transport device, or placed in a transport container to reach the second location. In this manner, there is a discrete break in the processing to allow for time associated with sample transport.

In another embodiment herein, separator gel(s) can also be included in the sample containers such that the gels will separate cell-free fractions of whole blood from the cellular or other solid or semi-solid portions of the sample. Such a gel or other similar separator material may be included in the sample container prior to, during, or after sample has been introduced into the sample container. The separator material may have a density between that of the cells and solution components, so that the material separates the sample components by flowing to a position between the solution and non-solution sample layers during separation such as by centrifugation. Following centrifugation, the separator material stops flowing and remain as a soft barrier between the layers. In some embodiments, the separator material can be further processed to harden into a more rigid barrier. In one non-limiting example, the separator material may be a UV-curable material such as but not limited to thixotropic gel of sorbitol-based gelator in a diacrylate oligomer. The sample container may have the entire vessel or optionally, on that portion with the UV-curable material exposed to UV light for a period of time such as but not limited to 10 to 30 seconds to harden the material. Such hardening may involve cross-linking of material in the UV-curable material. Optionally, the UV curable material may be used in conjunction with traditional separator gel material such that only one side (the solution side or the solid side) is in contact with the UV cured material. Optionally, the UV cured material may be used with a third material such that the UV cured material is between two separator materials and is not in direct contact with the solution and non-solution portions of the sample.

In one embodiment described herein, s method is provided for obtaining a sample from a subject, said subject having a body part, comprising: placing a novelty exterior over a sample collection device; and obtaining a sample from said body part of said subject using said sample collection device with the novelty exterior. Optionally, the method further comprises creating a wound site on said body part for sample to gather on a surface of the body part for sample collection. Optionally, the sample collection device comprises a capillary channel in fluid communication with a sample collection container. Optionally, the method comprises distracting the subject with the novelty exterior while obtaining the sample. Optionally, the novelty exterior covers a portion but not all of the sample collection device. Optionally, the method comprises removing a sterility barrier from at least one inlet on the sample collection device. Optionally, the method comprises removing a sterility barrier covering at least one inlet and at least a pre-determined sterile zone on the sample collection device around said inlet. Optionally, the method comprises, after collecting the sample, removing only a portion of the sample collection device containing the sample while leaving behind a capillary collection portion. Optionally, the method comprises after collecting the sample, removing the novelty exterior from the sample collection device and giving it to the subject as a souvenir. Optionally, the method comprises after collecting the sample, removing the novelty exterior from the sample collection device, sterilizing all or some portion of the novelty exterior, and giving it to the subject as a souvenir. Optionally, the method comprises preparing a target location on a body part by warming of at least the target site on said body part with at least one warming device selected from the group consisting of: warming table; a warming plate; a fingertip warmer; an air-warmer; furniture for seating comprising a warming plate or other heating element; and a combination thereof.

In another embodiment, an assembly is provided comprising a sample collection device; and a novelty exterior coupled to the sample collection device. Optionally, the novelty exterior has an interior that defines a cavity for receiving the sample collection device. Optionally, the novelty exterior is configured to represent a fish-like structure to the subject. Optionally, the novelty exterior is configured to represent a train-like structure to the subject. Optionally, a method is provided for obtaining a sample from a subject, the subject having a body part, comprising: obtaining a sample from said body part of said subject using a sample collection device with a novelty exterior. Optionally, the device comprises a sample collection device with a novelty exterior.

Optionally, a method is provided comprising at least one technical feature from any of the embodiments herein. Optionally, a method is provided comprising at least any two technical features from any of the embodiments herein. Optionally, a device is provided comprising at least one technical feature from any of the embodiments herein. Optionally, device comprising at least any two technical features from any of the embodiments herein. Optionally, a system is provided comprising at least one technical feature from any of the embodiments herein. Optionally, a system is provided comprising at least any two technical features from any of embodiments herein. Other configurations that can be useful to distract or entertain a subject during the sample collection process can be used.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A to 1C shows various views of one embodiment as described herein.

FIGS. 2A to 2B shows perspective and side views of one embodiment as described herein.

FIG. 3A shows an exploded side view of one embodiment as described herein.

FIG. 3B shows a side view of one embodiment as described herein.

FIGS. 4A to 4C show cross-sectional side views of various embodiments as described herein.

FIGS. 5A to 5C show cross-sectional side views of various embodiments as described herein.

FIGS. 6A to 6C show cross-sectional side views of various embodiments as described herein.

FIGS. 7A to 7C show cross-sectional side views of various embodiments as described herein.

FIGS. 8A to 8C show side views of various embodiments as described herein.

FIG. 9A shows a cross-sectional side view of one embodiment as described herein.

FIGS. 9B to 9C show side views of various embodiments as described herein.

FIGS. 10A to 10B show side views of various embodiments as described herein.

FIGS. 11A to 11C show side views of various embodiments as described herein.

### DETAILED DESCRIPTION

Methods, devices, and systems for obtaining a biological sample from a subject, such as, e.g., blood, are disclosed, for example, in PCT Patent Application WO2014/039909, filed Sep. 6, 2013; in PCT Patent Application WO2014/145935, filed Mar. 17, 2014; and in PCT Patent Application WO2014/088606, filed Dec. 5, 2013, the disclosures of all of which patent applications are hereby incorporated by reference in their entireties.

Description and disclosure of examples of obtaining samples and of sample analysis methods, devices, and systems, including automated analysis devices, semi-automated analysis devices, and systems comprising such devices, which may be used with the methods and systems disclosed herein may be found, for example, in U.S. Pat. No. 8,088,593; U.S. Pat. No. 8,380,541; PCT/US2012/57155, filed Sep. 25, 2012; U.S. patent application Ser. No. 13/244,949, filed Sep. 26, 2011; and in U.S. application Ser. No. 13/244,946, filed Sep. 26, 2011; the disclosures of all which patents and patent applications are hereby incorporated by reference in their entireties.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a compound” may include multiple compounds, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for a sample collection device, this means that the sample collection device may or may not be present, and, thus, the description includes both structures wherein a device possesses the sample collection device and structures wherein sample collection device is not present.

As used herein, the terms “substantial” means more than a minimal or insignificant amount; and “substantially” means more than a minimally or insignificantly. Thus, for example, the phrase “substantially different”, as used herein, denotes a sufficiently high degree of difference between two numeric values such that one of skill in the art would consider the difference between the two values to be of statistical significance within the context of the characteristic measured by said values. Thus, the difference between two values that are substantially different from each other is typically greater than about 10%, and may be greater than about 20%, preferably greater than about 30%, preferably greater than about 40%, preferably greater than about 50% as a function of the reference value or comparator value.

As used herein, the term “point of service location” (POS) may include locations where a subject may receive a service (e.g. testing, monitoring, treatment, diagnosis, guidance, sample collection, ID verification, medical services, non-medical services, etc.), and may include, without limitation, a subject's home, a subject's business, the location of a healthcare provider (e.g., doctor), hospitals, emergency rooms, operating rooms, clinics, health care professionals' offices, laboratories, retailers [e.g. pharmacies (e.g., retail pharmacy, clinical pharmacy, hospital pharmacy), drugstores, supermarkets, grocers, etc.], transportation vehicles (e.g. car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, bodily fluid sample acquisition sites (e.g. blood collection centers), sites at or near an entrance to a location that a subject may wish to access, sites on or near a device that a subject may wish to access (e.g., the location of a computer if the subject wishes to access the computer), a location where a sample processing device receives a sample, or any other point of service location described elsewhere herein.

As used herein, a “sample”, or “biological sample”, or “clinical sample” refers to a sample of fluid, tissue, secretion, or excretion obtained from a subject. A clinical sample may be a sample of blood, serum, plasma, saliva, sputum, urine, gastric fluid, digestive fluid, tears, sweat, stool, semen, vaginal fluid, interstitial fluid, fluid derived from tumorous tissue, ocular fluids, mucus, earwax, oil, glandular secretions, spinal fluid, skin, cerebrospinal fluid from within the skull, tissue, fluid or material from a nasal swab, a throat swab, a cheek swab, or nasopharyngeal wash, biopsy fluid or material, placental fluid, amniotic fluid, cord blood, lymphatic fluids, cavity fluids, pus, microbiota obtained from a subject, meconium, breast milk, or other secretion or excretion. A sample may be a breath sample, a hair sample, a fingernail sample, or other sample.

Biological samples may include nasopharyngeal wash, or other fluid obtained by washing a body cavity or surface of a subject, or by washing a swab following application of the swab to a body cavity or surface of a subject. Nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone. The sample may be obtained from a human or animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport.

Thus, as used herein, a “sample” may be but is not limited to a blood sample, or a portion of a blood sample, may be of any suitable size or volume, and is preferably of small size or volume. In some embodiments of the assays and methods disclosed herein, measurements may be made using a small volume blood sample, or no more than a small volume portion of a blood sample, where a small volume comprises no more than about 5 mL; or comprises no more than about 3 mL; or comprises no more than about 2 mL; or comprises no more than about 1 mL; or comprises no more than about 500 μL; or comprises no more than about 250 μL; or comprises no more than about 100 μL; or comprises no more than about 75 μL; or comprises no more than about 50 μL; or comprises no more than about 35 μL; or comprises no more than about 25 μL; or comprises no more than about 20 μL; or comprises no more than about 15 μL; or comprises no more than about 10 μL; or comprises no more than about 8 μL; or comprises no more than about 6 μL; or comprises no more than about 5 μL; or comprises no more than about 4 μL; or comprises no more than about 3 μL; or comprises no more than about 2 μL; or comprises no more than about 1 μL; or comprises no more than about 0.8 μL; or comprises no more than about 0.5 μL; or comprises no more than about 0.3 μL; or comprises no more than about 0.2 μL; or comprises no more than about 0.1 μL; or comprises no more than about 0.05 μL; or comprises no more than about 0.01 μL.

As used herein, a “small volume” refers to a volume of less than about 1 mL, or less than about 500 μL, or less than about 250 82 L, or less than 150 μL, or less than about 100 μL, or less than about 50 μL, or less than about 25 μL, or less. In particular embodiments, a small volume, such as a “finger-stick” volume, may comprise less than about 250 μL, and typically comprises less than 150 μL, or less than about 100 μL, or less than about 50 μL, or less than about 25 μL, or less.

As used herein, a “short period of time” refers to a period of time of about 5 hours or less, or about 4 hours or less, or about 3 hours or less, or about 2 hours or less, or about 1 hour or less, or about 50 minutes or less, or about 40 minutes or less, or about 30 minutes or less, or about 20 minutes or less, or about 10 minutes or less, or about 5 minutes or less. A short period of time may be determined with respect to an initial time; the initial time may be the time at which a sample analysis began; the initial time may be the time at which a sample is inserted into a device for the analysis of the sample; the initial time may be the time at which a sample was obtained from a subject.

It should be understood that although this indication may be by way of a visual indication, other indication methods such as audio, vibratory, or other indication methods may be used in place of or in combination with the indication method. The indicators may be on at least one of the containers. There may be variations and alternatives to the embodiments described herein and that no single embodiment should be construed to encompass the entire invention.

Optionally, the cap or other covering for the sample collection device may attach to the collection body of the sample collection device using any technique known or later developed in the art. For instance, the cap may be snap fit, twist on, friction-fit, clamp on, have magnetic portions, tie in, utilize elastic portions, and/or may removably connect to the collection unit body. Optionally, the cap may form a fluid-tight seal with the sample collection device body. The cap may be formed from an opaque, transparent, or translucent material.

The collection unit body may be permanently affixed to the support or may be removable with respect to the support. In some instances, the collection unit body and the support may be formed of a single integral piece. Alternatively, the collection unit body and support may be formed from separate pieces. Optionally, during the operation of the device, the collection unit and support do not move relative to one another.

In some examples, the collection unit body may have a circular, elliptical, triangular, quadrilateral (e.g., square, rectangular, trapezoidal), pentagonal, hexagonal, octagonal, or any other cross-sectional shape. The cross-sectional shape may remain the same or may vary along the length of the collection unit body. In some instances, the collection unit body may have a cross-sectional area of less than or equal to about 10 cm², 7 cm², 5 cm², 4 cm², 3 cm², 2.5 cm², 2 cm², 1.5 cm², 1 cm², 0.8 cm², 0.5 cm², 0.3 cm², or 0.1 cm². The cross-sectional area may vary or may remain the same along the length of the collection unit body 120. The collection unit body may have a length of less than or equal to about 20 cm, 15 cm , 12 cm, 10 cm, 9 cm, 8 cm, 7 cm, 6 cm, 5 cm, 4 cm, 3 cm, 2 cm, 1 cm, 0.5 cm, or 0.1 cm. The collection unit body 120 may have a greater or lesser length than the cap, support or base, or an equal length to the cap, support, or base. There may be variations and alternatives to the embodiments described herein and that no single embodiment should be construed to encompass the entire invention.

Although the channels may have any shape or size, some embodiments are configured such that the channel exhibits a capillary action when in contact with sample fluid. In some instances, the channel may have a cross-sectional area of less than or equal to about 10 mm², 7 mm², 5 mm², 4 mm², 3 mm², 2.5 mm², 2 mm², 1.5 mm², 1 mm², 0.8 mm², 0.5 mm², 0.3 mm², or 0.1 mm². The cross-sectional size may remain the same or may vary along the length. Some embodiments may tailor for greater force along a certain length and then less in a different length. The cross-sectional shape may remain the same or may vary along the length. Some channels are straight in configuration. Some embodiments may have curved or other shaped path shapes alone or in combination with straight portions. Some may have different orientations within the device body 120. For example, when the device is held substantially horizontally, one or more channels may slope downward, slope upward, or not slope at all as it carries fluid away from the initial collection point on the device.

In some instances, a plurality of channels may be provided. In some embodiments, one channel splits into two or more channels. Optionally, some channels split into an even larger number of channels. Some channels may include a control mechanism such as but not limited to a valve for directing flow in the channel(s). At least a portion of the channels may be substantially parallel to one another. Alternatively, no portion of the channels need be parallel to one another. In some instances, at least a portion of the channels are not parallel to one another. Optionally, the channels may be slightly bent. Optionally, channels may have one cross-sectional area at one location and a smaller cross-sectional area at a different location along the channel. Optionally, channels may have one cross-sectional area at one location and a larger cross-sectional area at a different location along the channel. For some embodiments of the Y design, it may be desirable that the channels would have vents placed appropriately to define the sample for each vial such that there would not be sample pulled or cross contamination from other channels.

The containers may be sized to contain a small fluid sample. In some embodiments, the containers may be configured to contain no more than about 5 ml, 4 ml, 3 ml, 2 ml, 1.5 mL, 1 mL, 900 uL, 800 uL, 700 uL, 600 uL, 500 uL, 400 uL, 300 uL, 250 uL, 200 uL, 150 uL, 100 uL, 80 uL, 50 uL, 30 uL, 25 uL, 20 uL, 10 uL, 7 uL, 5 uL, 3 uL, 2 uL, 1 uL, 750 nL, 500 nL, 250 nL, 200 nL, 150 nL, 100 nL, 50 nL, 10 nL, 5 nL, or 1 nL. The containers may be configured to contain no more than several drops of blood, a drop of blood, or no more than a portion of a drop of blood.

Optionally, the containers may contain a cap. The plug may be configured to fit over an open end of the container. The cap may block the open end of the container. The cap may fluidically seal the container. The cap may form a fluid-tight seal with the container body. For example, the cap may be gas and/or liquid impermeable. Alternatively, the cap may permit certain gases and/or liquids to pass through. In some instances, the cap may be gas permeable while being liquid impermeable. The cap may be impermeable to the sample. For example, the cap may be impermeable to whole blood, serum or plasma. In some instances, a portion of the cap may fit into a portion of the container body. The cap may form a stopper with the container body. The cap may include a lip or shelf that may hang over a portion of the container body. The lip or shelf may prevent the cap from sliding into the container body. In some instances, a portion of a cap may overlie a top and/or side of the container body. Any description herein of containers may be applied in combination with the sample collection device. Optionally, some embodiments may include an additional part in the vessel assembly such as cap holder. In one embodiment, the purpose of the cap holder is to maintain a tight seal between the cap and container. In one embodiment, the cap holder engages an attachment, lip, indentation, or other attachment location on the outside of the container to hold the cap in position. Optionally, some embodiments can combine the function of both the cap and the cap holder into one component.

The cap may be formed of a material that may prevent sample from passing through in the absence of a penetrating member. The cap may be formed from a single solid piece. Alternatively, the cap may include a slit, opening, hole, thin portion, or any other feature that may accept a penetrating member. A slit or other opening may be capable of retaining sample therein, when the penetrating member is not in the slit or opening, or when the penetrating member is removed from the slit or opening. In some instances, the cap may be formed from a self-healing material, so that when a penetrating member is removed, the opening formed by the penetrating member closes up. The second end of the channel may be a penetrating member that may pass through the cap and into the interior of the container. In some embodiment, it should be clear that the penetrating member may be hollow needles that allow sample to pass through, and not just needles for piercing. In some embodiments, the piercing tip can be a non-coring design such as but not limited to a tapered cannula that pierces without coring the cap material.

In some embodiments the inner surface of the channel and/or other surfaces along the fluid pathway such as but not limited to the sample inlet to the interior of a sample collection vessel may be coated with a surfactant and/or an anti-coagulant solution. The surfactant provides a wettable surface to the hydrophobic layers of the fluidic device and facilitate filling of the metering channel with the liquid sample, e.g., blood. The anti-coagulant solution helps prevent the sample, e.g., blood, from clotting when provided to the fluidic device. Exemplary surfactants that can be used include without limitation, Tween, TWEEN®20, Thesit®, sodium deoxycholate, Triton, Triton®X-100, Pluronic and/or other non-hemolytic detergents that provide the proper wetting characteristics of a surfactant. EDTA and heparin are non-limiting anti-coagulants that can be used. In one non-limiting example, the embodiment the solution comprises 2% Tween, 25 mg/mL EDTA in 50% Methanol/50% H20, which is then air dried. A methanol/water mixture provides a means of dissolving the EDTA and Tween, and also dries quickly from the surface of the plastic. The solution can be applied to the channel or other surfaces along the fluid flow pathway by any technique that will ensure an even film over the surfaces to be coated, such as, e.g., pipetting, spraying, printing, or wicking

It should also be understood for any of the embodiments herein that a coating in the channel may extend along the entire path of the channel. Optionally, the coating may cover a majority but not all of the channel. Optionally, the coating may cover only a portion of the channel. Optionally, some embodiments may not cover the channel in the areas nearest the entry opening to minimize the risk of cross-contamination, wherein coating material from one channel migrates into nearby channels by way of the channels all being in contact with the target sample fluid at the same time and thus having a connecting fluid pathway. Optionally, some embodiments may have the coating in pattern configuration in the channel.

Although embodiments herein are shown with two separate channels in the sample collection device, it should be understood that some embodiments may use more than two separate channels. Optionally, some embodiments may use less than two fully separate channels. Some embodiments may only use one separate channel. Optionally, some embodiments may use an inverted Y-channel or other split channel configuration that starts as one channel and then splits into two or more channels. Any of these concepts may be adapted for use with other embodiments described herein.

It should be understood that embodiments in this disclosure may be adapted to have one or more of the features described below.

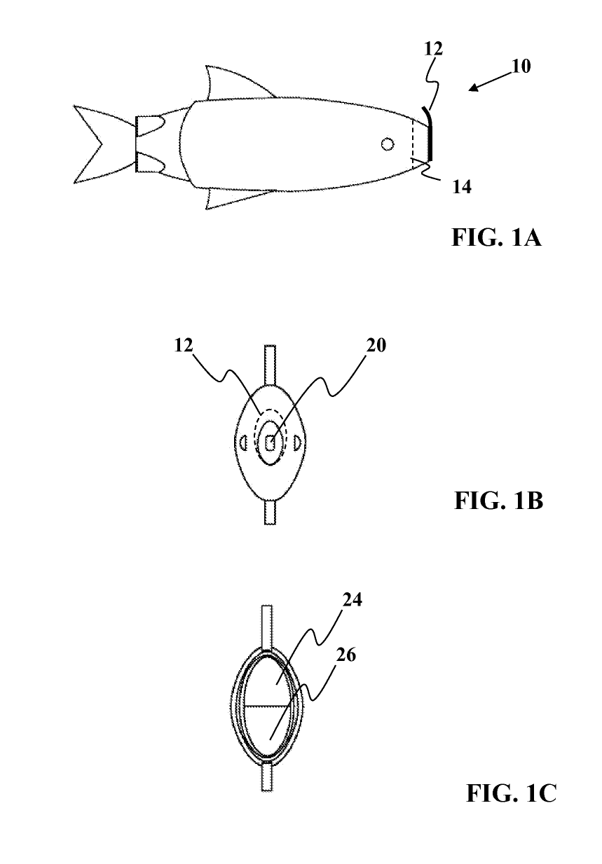

Referring now to FIG. 1, one embodiment of a novelty-exterior, sample collection device will now be described. The sample collection device can be useful for blood collection, which may be used to collect such sample from a target area on the subject. It should be understood that the part of their body targeted for obtaining a sample may include a hand, arm, foot, earlobe, fore-arm, or other part of the body. In embodiments of methods for obtaining a blood sample from a subject, blood may be obtained from a small skin puncture at a site on the subject that may or may not have been warmed to increase blood flow. Such a small skin puncture may be, e.g., a small skin puncture on a finger, such as on a fingertip; a small skin puncture on a toe, such as on or near a tip of a toe; a small skin puncture on a foot, such as on or near the heel; or other small skin puncture. A small skin puncture is on a finger may be termed a “finger-stick”; the term finger-stick may also be used to describe the volume of a sample, i.e., a finger-stick volume is one, two, or a few drops (e.g., about 50 μL to about 300 μL, or about 75 μL to about 250 μL, or about 80 μL to about 150 μL). In one embodiment, blood from such a wound is typically capillary blood. Optionally, blood collected for certain other sizes may comprise a different mixture of capillary or other blood types.

FIG. 1A shows one non-limiting embodiment of a novelty-exterior, sample collection device 10 wherein the novelty exterior has a likeness of an animal such as but not limited to a fish, a clown fish, an eel, a starfish, a mollusk, a worm, a dog, a cat, a pig, a beaver, a horse, bear, possum, kitten, panda, koala, duckbill platypus, turtle, snake, alligator, tapir, marmot, fictional animal, cartoon animal, extinct animal, a troll, a color changing troll, dragon, dinosaur, space alien, or other animal. This exterior form may make the blood extraction process proceed more smoothly, particularly for, but not limited to pediatric subjects. In one non-limiting example, the novelty-exterior, sample collection device 10 can be a useful distraction to take the pediatric subjects attention away from the procedure/make it seem innocuous, and/or it can be a reward for cooperating with the blood or other sample extraction procedure. One non-limiting example uses a jellyfish for novelty exterior, but other animals such as a squid, a fish, a bear, a pig, or others are not excluded. Optionally, other embodiments may have a non-animal, novelty exterior. Non-animal toy-like exterior appearance for the novelty exterior, sample collection device 10 can also be used such as but not limited to a rocket, an airplane, a car, a race car, a train, a train locomotive, a ship, a space ship, a ghost, or other elongate toy-like shape. Some may form the exterior of plush material that provides a stuffed animal type feel. Optionally, others may use a combination of hard and soft materials. Optionally, still others may use primarily hard materials for forming the toy-like exterior. In embodiments, the novelty exterior comprises a hard surface that is easily cleaned, and that may be sterilized. Such a surface may be or include, for example, plastic, glass, hard rubber, acrylic, polyvinylchloride, polyethylene, polyurethane, other polymers, and other materials. In embodiments, the novelty exterior may comprise a soft or padded surface that is easily cleaned, and that may be sterilized. Such a surface may be or include, for example, cloth, rubberized cloth, soft rubber, and other materials. Optionally, some embodiments may combine one or more of the foregoing. Optionally, some embodiments may have color-temperature change material wherein the color of the novelty exterior will change when temperature changes, either due to the sample being collected, the user handling the sample collection device, or other temperature change causing event.

FIG. 1A also shows that in at least one embodiment herein, there may be at least one sterility barrier 12 coupled to the collection device 10 in a manner that prevents external contaminants from entering any channel(s) or fluid pathways inside the collection device 10. By way of non-limiting example, the barrier may be a metal foil, polymer, metal foil/polymer combination, or other materials suitable for a sterility barrier. Optionally, some embodiments may use a peelable barrier. Optionally, some embodiments may use a burstable barrier this is not peeled or fully peeled before being use. Optionally, the sterility barrier comprises a material that can easily be broken when an edge of a device applies a force thereto. Optionally, the sterility barrier alone or in combination with other barriers may be used to create a sterile environment about at least the tip of the sample collection device 10 prior to use. Optionally, the sterility barrier may be made of a variety of materials such as but not limited to metallic foil, aluminum foil, paper, polymeric material, or laminates combining any of the foregoing.

Optionally, some embodiments may have the barrier 12 covering primarily a distal end surface of the collection device 10. Optionally, some embodiments may be configured to cover at least a portion of the distal end and at least a portion of a side surface of the collection device near the distal end of the collection device 10. In this non-limiting example, both the end surface and relevant side surface(s) up to line 14 (shown in phantom) of the collection device 10 are kept sterile prior to use. In some embodiments, the line 14 is about 1 mm from the distal end of the collection device 10. Optionally, the line 14 is about 2 mm from the distal end of the collection device 10. The position and/or shape of line 14 are merely exemplary and other positioning and/or shape of the line 14 such as wavy, angled, or the like are not excluded. In some embodiments, the surface of the collection device 10 may not be painted or otherwise decorated so that the surface can be kept sterile. In such an embodiment, the undecorated zone may be used with embodiments with the distal-end-only barrier 12, the distal end and side surface barrier, or other barrier configurations. Optionally, the undecorated zone may extend beyond the line 14, may not be a straight line but follow some other shape, may be a shape that is not the shape of line 14, or may be some other or combination of configurations. By of way of non-limiting example, the barrier 12 may be coupled to the collection device 10 while both are in a sterile environment.

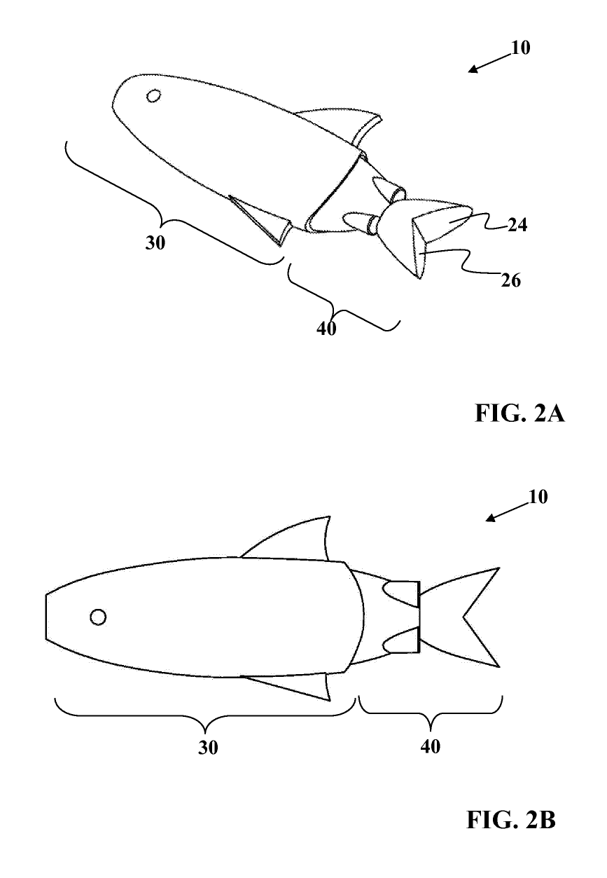

Optionally, one embodiment may use multiple layer sterility barriers. It should be understood that the configuration with multiple sterility barriers may be adapted for use with any embodiment of the present invention. In one non-limiting example, the sterility barrier is a two layer sterility barrier. Such a multiple layer sterility barrier can be used for any of the embodiments discussed herein. Optionally, the multiple layer sterility barrier can also include three or more layers. Optionally, some embodiments may have a sterility barrier at a distal end and at least one sterility barrier on a proximal end. As seen in FIG. 2A, some embodiments may have a sample collection device that has at least two portions 30 and 40, wherein at least one end of each of the portions includes at least one sterility barrier. In one embodiment, one portion may have a sterility barrier on both a distal end and a proximal end, while the other portion only has a sterility barrier on one end such as but not limited to a distal end. Optionally, some embodiments may include at least two barriers on each portion. Optionally, some embodiments, instead of using a removable barrier, may instead place each portion in its own sterility bag or container such that there is no barrier attached to the device.

Optionally, a distal end surface of the collection device 10 may be coated or formed of a hydrophobic material to direct sample in certain direction(s). Optionally, the collection device 10 may be coated with hydrophilic material. Optionally, some embodiments may use a combination both of the foregoing. Optionally, some embodiments may use a combination both of the foregoing in a patterned manner.

FIG. 1B shows that in the present embodiment, a novelty exterior, sample collection device 10 includes at least one opening 20 for receiving at least one sample therein. Optionally, some embodiments may have at least two openings 20 sized for sample collection. Optionally, some embodiments may have multiple openings 20, with at least one of the openings on a different surface of the novelty-exterior, sample collection device 10 relative to one other opening. Optionally, some embodiments may have the opening 20 on a bottom or other side of the novelty exterior, sample collection device 10. Some fish such as but not limited to cat fish, have that type of side-located opening.

FIG. 1B also shows that, in one non-limiting example, the opening 20 is positioned at or near a distal end of the sample collection device. In this manner, the opening 20 can be easily positioned by a technician over at least a portion of the pad portion of the fingertip. In this manner, the tissue to be targeted which is typically not on the side of the finger nail, can be heated to a desired temperature to increase blood flow. This pad portion of the fingertip is typically on the palm side of the finger and may include one or more sides of the fingertip. Although opening 20 is shown as having a circular cross-sectional shape, it should be understood that other cross-sectional shapes may be used, such as but not limited to polygonal, rectangular, triangular, square, oval, single or multiple combinations of shapes, or other shapes. Also, it should be understood that the sterility barrier can also have a shape, when viewed from the distal end, such as but not limited to polygonal, rectangular, triangular, square, oval, single or multiple combinations of shapes, or other shapes.

A sample obtained by such methods may be a small sample. For example, such a sample may be obtained from a finger-stick, and may comprise a few drops, or two drops, or one drop, of blood obtained from a small lancet puncture in the skin of the subject. The sample, such as a blood sample, may be obtained from the subject following warming a body part (e.g., a fingertip, or finger, of fingers, or hand) of subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

FIG. 1C shows a view of the proximal end of the collection device with tail surfaces 24 and 26. Optionally, some embodiments can configure the proximal end to be a flat or other shaped surface that allows the base portion 40 to be self-standing when removed from the collection portion 30. In one non-limiting example, base portion 40 is self-standing in a vertical orientation for the containers 50 therein. Optionally, base portion 40 is self-standing in a horizontal orientation for the containers 50 therein. Optionally, base portion 40 is self-standing in some other orientation for the containers 50 therein.

In some embodiments, the novelty exterior may provide a calming feature such as but not limited to a calming scent, which may be provided by pads, tapes, oils, aerosols, or other means present on the novelty exterior. Such calming scents may include perfumes, aromas, and other scents and scented materials, whether natural, artificial, or combinations thereof. Calming scents include, e.g., vanilla, rose scent, lavender, coconut, marjoram, chamomile, lilac, citrus scents, and others. Such scents may be provided by flowers (e.g., rose, jasmine, geraniums, and others) or plants (e.g., sage, mint, rosemary, and others) present in the sample collection room or placed in or near sources of airflow into the sample collection room. Optionally, some embodiments may provide optical features such as but not limited to one or more LED or other light features to distract a subject during the collection process. Some embodiments may include a small LCD or other screen to display a message, a name, or other information that can distract a subject from the collection procedure.

In some embodiment, the novelty exterior is provided separate from the sample collection device. Optionally, when combined, the novelty exterior and the sample collection device can form the novelty exterior sample collection device. Depending on age, gender, or other factor, the subject or the medical provider can select the novelty exterior at, before, or during the collection process based on the desires of the subject. Optionally, some embodiments may be configured so that a subject can select the novelty exterior that the subject desires to use with the sample collection device for their collection procedure.

Optionally, a surface of a novelty exterior and/or the sample collection device may have one or more thermal controlled sites wherein the temperature of the target site on the patient (e.g., a finger) may be brought to a desired temperature. By way of example and not limitation, embodiments may heat distal end surface and/or adjacent side surface(s) of the collection device and/or novelty exterior to improve blood flow and thus blood yield from a finger-stick. For example, the distal end may have thermal control areas to increase blood flow to the target area and thus increase the speed with which sufficient blood or other bodily fluid can be drawn from the subject. The heating derived from the heating element may bring the target tissue to between about 40° C. to about 50° C. In embodiments, the heating derived from the heating element is configured to bring target tissue to within a temperature range of between about 40° C. to about 44° C. In embodiments, the heating derived from the heating element brings target tissue to within a temperature range of between about 41° C. to about 43° C. In embodiments, the heating derived from the heating element brings target tissue to a temperature of about 42° C. In embodiments, the heating derived from the heating element brings target tissue to within a temperature range of between about 44° C. to about 47° C. In one embodiment, the temperature is sufficient to increase blood flow to yield about 120 uL of sample. In one embodiment, the temperature is sufficient to increase blood flow to yield about 130 uL of sample. In one embodiment, the temperature is sufficient to increase blood flow to yield about 140 uL of sample. In one embodiment, the temperature is sufficient to increase blood flow to yield about 150 uL of sample. Optionally, the thermal controlled site is a shaped surface is contoured to match that of the target site on a patient.

It should be understood that the novelty exterior may be textured and/or finished in a manner that contributes to the interest and/or distraction aspect of the novelty exterior. By way of non-limiting example, if the novelty exterior is a fish, then the exterior may be of a texture to suggest or feel like fish scales. Optionally, if the novelty exterior is a bird, the novelty exterior may be configured to include feathers or other bird like features attached to the exterior, albeit, the location of the features may be sufficiently away from the sample collection end so as not to interfere with sample collection. Accordingly, the novelty exterior can be embellished with additional elements, features, texture(s), 3D features, or other element based on the theme of the novelty exterior.

In embodiments, a novelty exterior may be placed over a substantial portion of a sample collection device, or over all of a sample collection device, in a manner similar to the manner in which the finger of a glove covers a finger. A sample collection device may be made from, or may include (e.g., as an inner lining) an absorbent material. A sample collection device may include perfume or other odorant. A sample collection device may include features which may suggest a face, or arms, or hair, or other features which may be amusing, or diverting, or comforting, to a subject (e.g., a child) who has had a blood sample taken from a finger. A sample collection device may include writing, or symbols, or other markings which may provide amusement, diversion, or comfort, or which convey a message or reminder to a subject. In embodiments, a sample collection device may identify the location or enterprise which performed or collected the sample. In embodiments, a sample collection device may include a commercial message, or may include symbols or other markings which convey a commercial message.

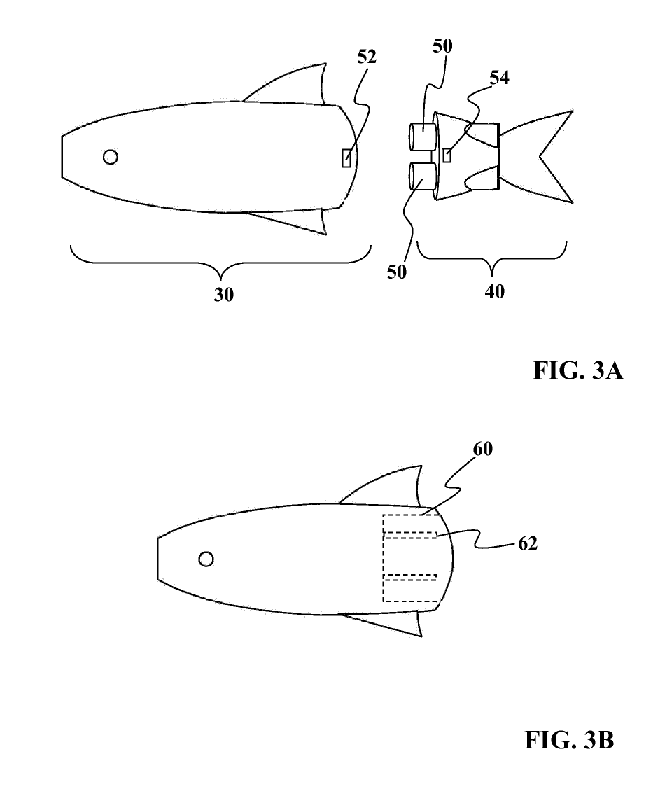

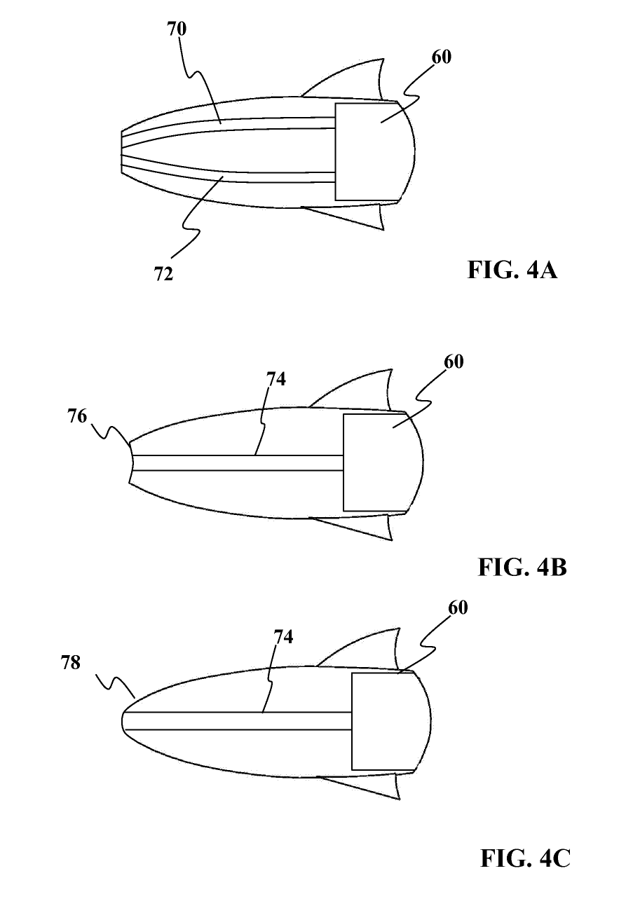

Referring now to FIGS. 2A and 2B, still further views of the novelty exterior, sample collection device 10 are shown. FIG. 2A shows that in this non-limiting example, there collection device 10 comprises a collection portion 30 and a base portion 40. In at least one embodiment, the collection portion 30 and base portion 40 may be releasably coupled together and may be separable as seen in FIG. 3A. In this manner, the sample can be contained in the base or a portion of the base for storage and/or transport. In this manner, the collection portion 30 may be discarded, resulting in a much smaller portion 40 to handle for processing, storage, and/or transport. Although this embodiment shows that there may be at least two portions to the sample collection device 10, it should be understood that some embodiments can have more portions, some or all of which have the novelty exterior. Optionally, some embodiments only have a single portion. Optionally, some embodiments may only have a single portion with a novelty exterior but may have one or more other portions that have a non-novelty exterior.

FIGS. 1A and 2B show that in at least one embodiment, both the left and the right sides of the exterior of the sample collection device 10 are substantially similar in configuration. Optionally, some embodiments may have slight variations on each side such as but not limited to different coloring, fin shape, texture, or other exterior feature. Optionally, some embodiments may be configured to have substantially similar shape, similar silhouette, or the like, but have very different coloring, texture, expression, or the like. For example, some embodiments may have the sample collection device 10 designed to have a day-time look on one side and a night time look on the other side. Yet another embodiment may be configured so that one side shows one emotion or other messaging, such as but not limited to a happy fish while the other side shows another emotion such as but not limited to an angry fish. Other variations between one side and the other side are not excluded.

Referring now to FIG. 3A, an exploded side view is shown of the collection portion 30 separated from the base potion 40. FIG. 3A shows that the base 40 may house or at least partially house one or more sample containers 50 that may be fixed to or be removable from the base 40. As seen in FIG. 3A, some embodiments may have more than one container 50 coupled to the base 40. Optionally, some embodiments may integrally form one or more of the containers 50 with the base 40. Optionally, some embodiments may have one or more containers integrally formed and one or more containers that are not integrally formed with the base 40.

As seen in FIG. 3A, some embodiments may include attachment features such as a mechanical latch, a hole-protrusion combination, a friction fit, a texture interference fit, or the like to releasably hold the collection portion 30 with the base 40. By way of non-limiting example, FIG. 3A shows that some embodiments may use an opening 52 designed to overlap with a protrusion 54 to create a locking configuration.

FIG. 3B shows that in at least some embodiments, there is at least one cavity 60 in the collection portion 30 that is sized to receive at least the containers 50 and/or base 40. In such an embodiment, the base 40 is movable from a first position in the collection portion to at least a second portion. In at least some embodiments, the first position is one where the base is secured to the collection portion 30, but has not necessarily created a pathway from at least one channel in the collection portion 30 with the container(s) 50. In at least some embodiments, the second position is one where a pathway from at least one channel in the collection portion 30 with the container(s) 50, such as but not limited to when at least one needle 62 on the collection portion 30 engages and pierces a septum on container 50. Other configurations for sample collection can be found in U.S. Provisional Application Ser. No. 61/852,489 filed Mar. 15, 2013. Some embodiments may provide at least one or more sterility barriers on one end of the containers 50.

By way of non-limiting example, the cavity 60 of FIG. 3B can be configured to have one or more features that allow for interface with the base portion 40. In one non-limiting example, the cavity 60 can have a cross-sectional shape that in at least one portion, matches with a cross-sectional shape of the base portion 40. Optionally, some embodiments of the base portion 40 and the cavity 60 may include a track, rail, keyed rail, keyed track, keyed shaped, or other guide device that allows for the alignment of the base portion 40 for insertion into the device 10.

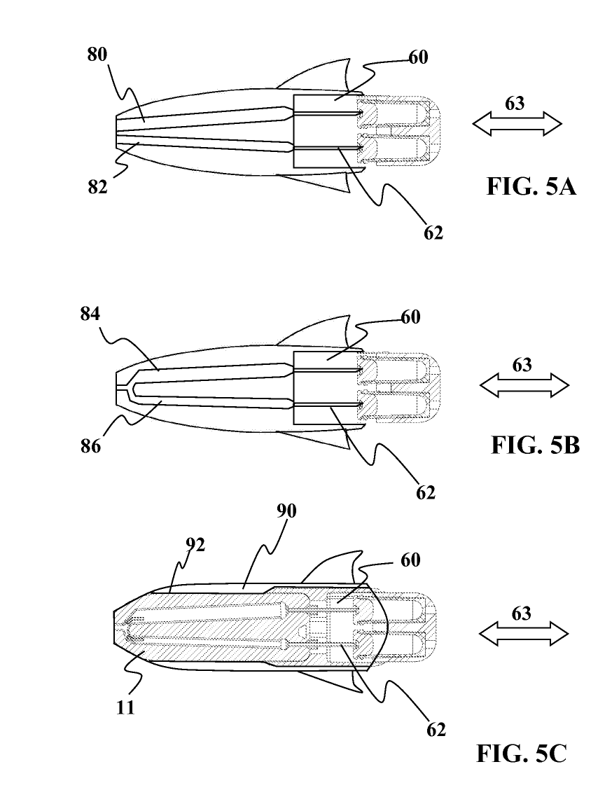

Referring now to FIGS. 4A through 4C, side-cross-sectional views of embodiments of sample collection devices 10 will now be described. FIG. 4A shows an embodiment having a plurality of curved collection channels 70 and 72 in the sample collection device 10. Some embodiments may have at least one curved and at least one non-curved channel. Optionally, some embodiments may have one channel larger in volume than the other channel. Optionally, there may be needles or other devices that couple to one end of the channels 70 and 72.

FIGS. 4B and 4C show embodiments wherein the collection device has a centrally positioned collection channel 74 and either a concave distal end surface 76 or convex distal end surface 78. The concave distal end surface 76 or convex distal end surface 78 can be selected to improve sample collection based on the tissue targeted for collection of the sample. Some may be better configured if the target is a finger or other rounded surface. Optionally, other may be better suited for collection from a flatter tissue surface. Optionally, the distal end surface may have a circular cross-sectional shape. Optionally, the distal end surface may have an oval cross-sectional shape. Optionally, the distal end surface may have a square cross-sectional shape. Optionally, the distal end surface may have a rectangular cross-sectional shape. Optionally, the distal end surface may have a polygonal cross-sectional shape. Optionally, the distal end surface may have a cross-sectional shape that combines one or more of the foregoing shapes. Some embodiments may be configured for left-hand sample capture. Some embodiments may be configured for right-hand sample capture. Some embodiments may be configured for sample capture regardless of which hand is targeted for sample capture or which hand is used by the technician to handle the sample collection device.

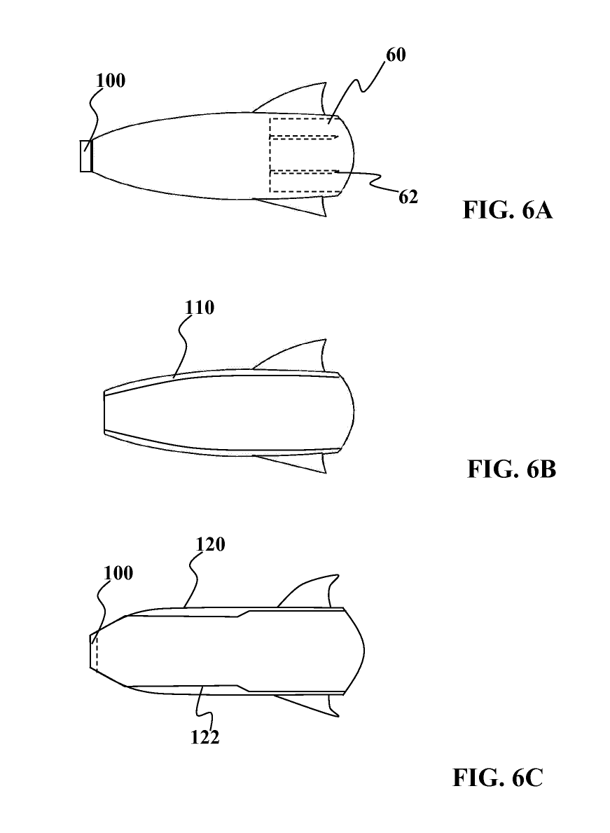

Referring now to FIGS. 5A through 5C, side-cross-sectional views of various embodiments of sample collection devices 10 will now be described.

FIG. 5A shows one embodiment wherein the sample collection device 10 includes at least two channels 80 and 82 in the sample collection portion of the device. Optionally, other embodiments may have three channels or more. Optionally, some embodiments may have a single channel. As seen in FIG. 5A, one channel 80 has a larger cross-sectional area than the other channel 82. Optionally, some embodiments may use channels of the same or substantially similar cross-sectional area, to the extent possible through manufacturing tolerances. Further details of embodiments of sample collection devices can be found in U.S. patent application Ser. No. 14/020,435 filed Sep. 6, 2013 and U.S. Patent Application 61/852,489 filed Mar. 15, 2013, both fully incorporated herein by reference for all purposes. In at least one embodiment, the containers are slidable relative to the collection body as indicated by arrow 63.

FIG. 5B shows one embodiment wherein the sample collection device 10 includes at least two channels 84 and 86 in the sample collection portion. As seen in FIG. 5A, one channel 84 has a larger cross-sectional area than the other channel 86. Optionally, some embodiments may use channels of the same or substantially similar cross-sectional area, to the extent possible through manufacturing tolerances. FIG. 5B also shows that, for at least some embodiments herein, multiple channels can be split from a single channel that leads from an inlet to the sample collection device. Further details of embodiments of sample collection devices can be found in U.S. patent application Ser. No. 14/020,435 filed Sep. 6, 2013 and U.S. Patent Application 61/852,489 filed Mar. 15, 2013, both fully incorporated herein by reference for all purposes.

FIG. 5C shows one embodiment wherein the novelty exterior such as but not limited to a sleeve, shell, or other outer structure is coupled to a sample collection device to form the sample collection device. This embodiment comprises using a sample collection device 11 that is a device for sample collection, but without the novelty exterior. In this manner, the sample collection device 11 can be manufactured in a uniform manner and then subsequently outfitted with the desired novelty exterior 90. This may differ from the embodiments of FIGS. 5A and 5B wherein the interior components of the sample collection device are integrally formed with the novelty exterior in a manner where they are not designed to be separate parts that are joined together. Further details of embodiments of sample collection devices can be found in U.S. patent application Ser. No. 14/020,435 filed Sep. 6, 2013 and U.S. Patent Application 61/852,489 filed Mar. 15, 2013, both fully incorporated herein by reference for all purposes.

FIG. 5C comprises an embodiment with a formed cavity that is configured to mate with at least a portion of the sample collection device. There may be an inner surface 92 shaped to conform with at least one portion of the collection unit 11. Some embodiment may have the inner surface 92 be textured, bumped, or otherwise shaped to allow for engagement between the novelty exterior 90 and the sample collection device. Some embodiments may have protrusion(s), indentation(s), and/or other feature(s) to couple the novelty exterior to the sample collection device. Optionally, other embodiments may be use glue, adhesive, or other attachment material alone or in combination with shaped features to secure the shaped exterior to the sample collection device.

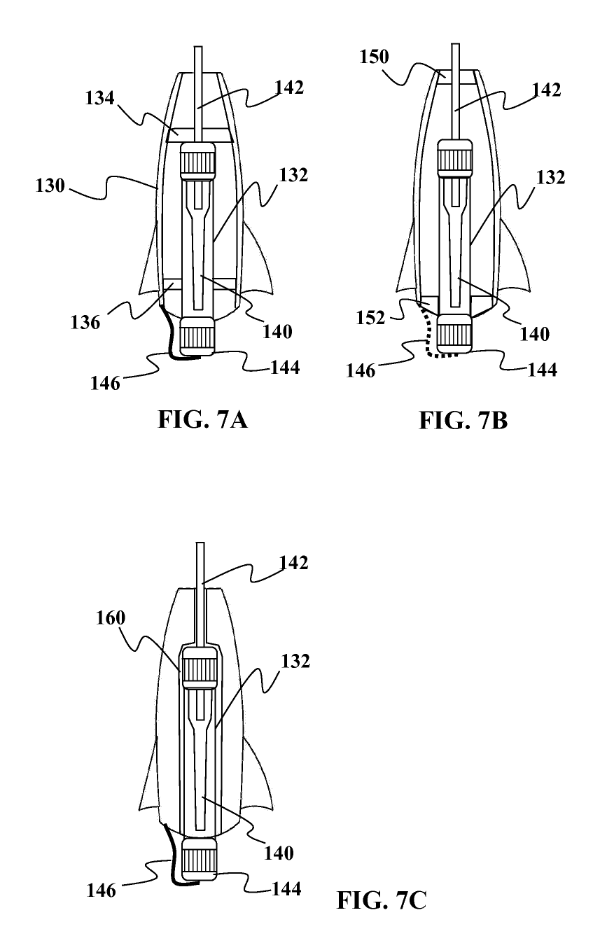

Referring now to FIGS. 6A to 6C, still further embodiments of the sample collection device will now be described. FIG. 6A shows a side view of one embodiment of a sample collection portion of the device 10, wherein there is a sterile portion 100 located about a distal end of the collection device. In one embodiment, the sterile portion 100 is not decorated with any portion of the novelty exterior. Optionally, one embodiment may have a sterile portion 100 with at least a portion with a novelty exterior. In one embodiment, the sterile portion 100 includes at least the side surface area about 1 mm from the distal end surface of the collection device. Optionally, the sterile portion 100 comprises at least the side surface area about 2 mm from the distal end surface of the collection device. Optionally, the sterile portion 100 comprises at least the side surface area about 3 mm from the distal end surface of the collection device. In some embodiments, the sterility barrier may be configured to cover at least one end and at least a sterile portion 100.

FIG. 6B shows another embodiment wherein the novelty exterior 110 is a sleeve or other pull-over type covering that can be installed over the sample collection device 10. In the present embodiment, the shape of the interior cavity is substantially smooth. FIG. 6B also shows an embodiment wherein the thickness of the material is substantially consistent in thickness. Optionally, embodiments such as that of FIG. 6B may also have an interior without interior steps or ridges. Optionally, embodiments such as that of FIG. 6B may be configured so that the sample collection device can only be inserted in one direction, such as but not limited to the use of an exterior shell with one end having a larger opening and one end with a smaller opening sized too small to receive the sample collection device if inserted through the smaller opening.

FIG. 6C shows another embodiment wherein the novelty exterior 120 may be a pull-over type covering such as but not limited to a sleeve, shell, or the like that can be installed over the sample collection device 10. In the present embodiment, the interior cavity has a shaped interior 122 with edges and/or surfaces configured to match the contour of at least some portion of a sample collection device to be contained therein. By way of non-limiting example, the interior is shown to have a narrowed portion and widened portion to match the shape of select portions of the sample collection device. Optionally, embodiments such as that of FIG. 6C may be configured so that the sample collection device can only be inserted in one direction, such as but not limited to the use of an exterior shell with one end having a larger opening and one end with a smaller opening sized too small to receive the sample collection device if inserted through the smaller opening. Optionally, the sample collection device can be shaped to allow for insertion in only one direction into the novelty exterior 120.

Optionally, some embodiments may have scaffolding and/or other interior structures to hold the sample collection device in a desired position and/or orientation within the novelty exterior. Thus, in one embodiment, a novelty exterior is provided on at least one exterior surface while an internal portion defines at least one attachment interface with the sample collection device. The embodiments FIGS. 6B and 6C differ from the embodiments of prior figures in that they are sleeves or covers that go over a sample collection device. In the embodiments of prior figures, the sample collection device has a novelty exterior that is integral with and/or integrally formed with the sample collection device. In at least FIG. 6C, the interior surface of the novelty exterior 120 defines at least one interface with an exterior surface of the sample collection device that fits at least partially therein.

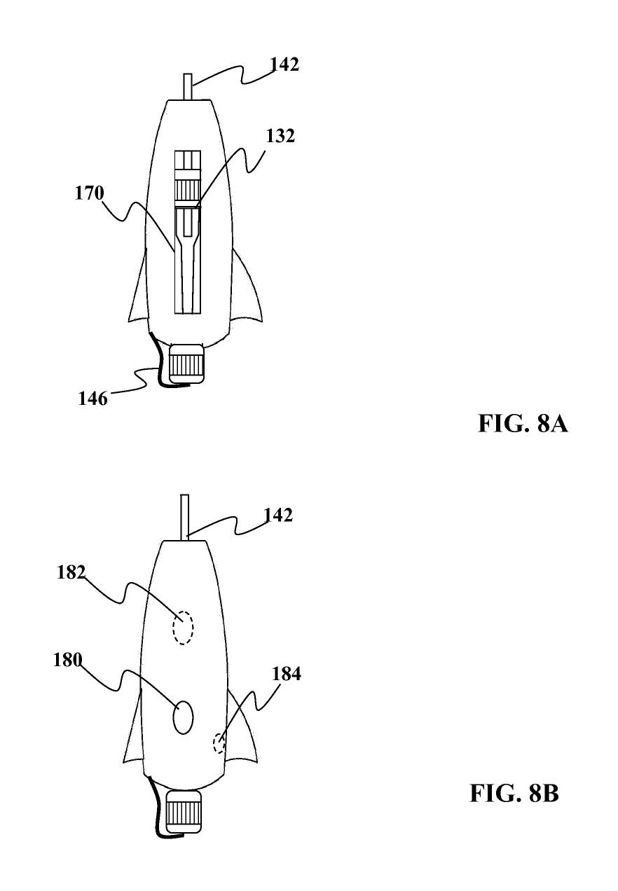

Referring now to FIGS. 7A and 7C, still further embodiments of a sample collection device 130 will now be described. As seen in FIG. 7A, this embodiment has a novelty exterior configured to contain a substantially cylindrical sample collection device 132. This embodiment has support features in the interior cavity of the novelty exterior such as but not limited to distal end interior stop 134 and a side mounted support 136 located closer to a proximal end of the sample collection device. This embodiment has a collection device 132 with interior 140, a capillary tube 142, and a removable end cap 144. FIG. 7A shows that there may be retaining member 146 that can hold the cylindrical sample collection device 132 in the novelty exterior. The retaining member 146 can be released so that the cylindrical sample collection device 132 can be removed. Optionally, it should be understood that other non-clip type retaining members such as screw caps, threaded attachments, or other retaining features may also be used.

In one embodiment, the novelty exterior can be made from an elastomeric material that can be placed around the circumference of the sample collection device. Optionally, elastomeric material may be provided that need not be provided around the entire circumference of the collection device. For example, one or more rubber balls or similar elastomeric protrusions may be provided at one or more intervals within the interior cavity of the novelty exterior. Optionally, the sample collection device may have one or more grooves into which one or more O-rings or protrusion on the novelty exterior may fit. Optionally, the sample collection device may have one or more grooves on its external surface into which one or more O-rings or other materials may fit.

Optionally, a high-friction and/or flexible material may be provided between a portion of the novelty exterior and/or the sample collection device. This may enable the novelty exterior to be press-fit onto the sample collection device, or for the sample collection to be press-fit into the novelty exterior. In some instances, both the sample collection device and novelty may have O-rings or similar materials. An O-ring may ensure a seal or connection between the sample collection device and the novelty exterior. Optionally, the pipette nozzle may have an internal shelf or flat back. The flat back may provide a physical stop to seat the novelty exterior in the appropriate location over the sample collection device. Optionally, other embodiments may have protrusions or other features that may act as a register to properly position the cover over the sample collection device.

Referring now to FIG. 7B, yet another embodiment is shown of the sample collection device with a novelty exterior. This embodiment varies the locations of the distal end interior stop 150 and a side mounted support 152. FIG. 7B shows that for this embodiment, the distal end interior stop 150 is in contact only with the collection channel, which in this embodiment comprises capillary tube 142. It should be understood that other embodiments may use devices other than a capillary tube to initiate collection of a sample. FIG. 7B also shows that the side mounted support 152 is located closer to a distal end of the collection device than in the embodiment of FIG. 7A.

Referring now to FIG. 7C, there is shown a shaped interior cavity which supports end and side surfaces of the cylindrical sample collection device 132 that are within the novelty exterior. The shaped interior cavity 160 has at least a narrowed portion which is configured about at least a portion of the collection channel, which in this embodiment comprises capillary tube 142. Although the capillary tube 142 herein may be a single lumen, single tube embodiment, other embodiments of the collection channel may optionally use a multi-lumen capillary tube and/or multiple capillary tubes. Some embodiments may combine the foregoing and include multi-lumen capillary tube and/or multiple single or multi-lumen capillary tubes. The shaped interior cavity 160 has at least a widened portion which is configured about at least a portion of the collection unit 132. Some embodiments may have an interior cavity 160 with a shape that substantially mimics the shape of the calibration unit 132. Optionally, some embodiments may have a shape wherein only select portions of the cavity shape is configured to match an outer shape of the collection device. Optionally, some embodiments may have only select portions of the cavity shape with material configured to contact an outer shape of the collection device. In one embodiment, the novelty exterior may be a bi-layer configuration with a first material on an outer portion and a second portion in the inner portion. In this manner, a configuration can be created where the exterior is hardened, while an interior has a softer material. Optionally, a configuration can be created where the exterior has a soft material, while an interior has a harder material relative to the material on the exterior.

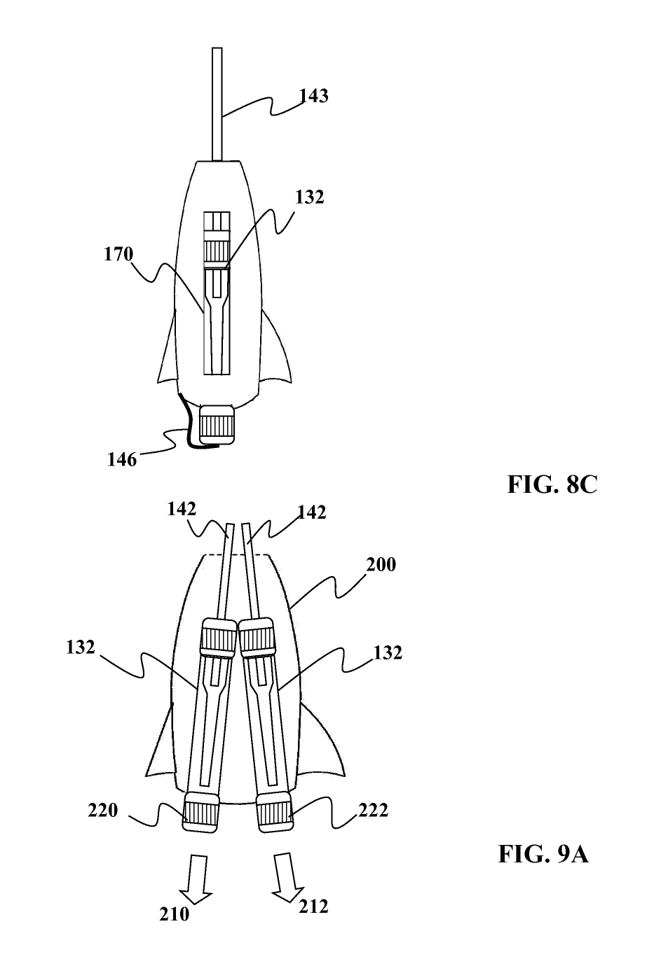

Referring now to FIGS. 8A and 8B, views of the exterior of various embodiments will now be shown. FIG. 8A shows an embodiment wherein there is an elongate window 170 to show fill level and/or other information about the sample collection device therein.

FIG. 8B shows an embodiment with one or more view openings. FIG. 8B shows that there is at least one embodiment of the sample collection device where there is a window 180 positioned to show if sample is being collected in the device. FIG. 8B also shows that some embodiments may have other windows 182 that may be positioned to show whether sample is being collected by the collection channel, which in this embodiment comprise capillary tube 142. In some embodiment, an indicator such as but not limited to visual or auditory indicator 184 can be mounted on the device

FIG. 8C shows yet another embodiment wherein the sample collection device has an elongated collection channel, which in this embodiment comprises a capillary tube 143 having a length sized to capture an additional volume due to the increased length of the tube 143 without decreasing the cross-sectional area of the channel therein. The capillary tube 143 is used to capture a minimum volume of sample in the collection channel prior to directing the fluid into the vessel portion of the collection device.

FIG. 8C shows that in this embodiment, the capillary tube 143 extends beyond the distal outer end surface of the novelty exterior by of a distance of at least 5 mm. Optionally, some embodiments may extend beyond the distal outer end surface of the novelty exterior by of a distance of at least 10 mm. Optionally, some embodiments may extend beyond the distal outer end surface of the novelty exterior by of a distance of at least 15 mm. Optionally, some embodiments may extend beyond the distal outer end surface of the novelty exterior by of a distance of at least 20 mm. Optionally, some embodiments may extend beyond the distal outer end surface of the novelty exterior by of a distance of at least 25 mm. Optionally, some embodiments may extend beyond the distal outer end surface of the novelty exterior by of a distance of at least 30 mm. Optionally, some embodiments may extend beyond the distal outer end surface of the novelty exterior by of a distance of at least 35 mm. Optionally, some embodiments may extend beyond the distal outer end surface of the novelty exterior by of a distance of at least 40 mm. Optionally, some embodiments may be configured wherein the length is measured based on the entire length of the collection channel. Optionally, some embodiments may have a collection channel length of at least 10 mm. Optionally, some embodiments may have a collection channel length of at least 15 mm. Optionally, some embodiments may have a collection channel length of at least 20 mm. Optionally, some embodiments may have a collection channel length of at least 25 mm. Optionally, some embodiments may have a collection channel length of at least 30 mm. Optionally, some embodiments may have a collection channel length of at least 35 mm. Optionally, some embodiments may have a collection channel length of at least 40 mm. Although this embodiment is shown with a retaining member 146, it should be understood that other embodiments may not use such a member. Optionally, some embodiments may use a press-fit type configuration to hold the sample collection device therein or some frictional engagement element to hold the collector therein.

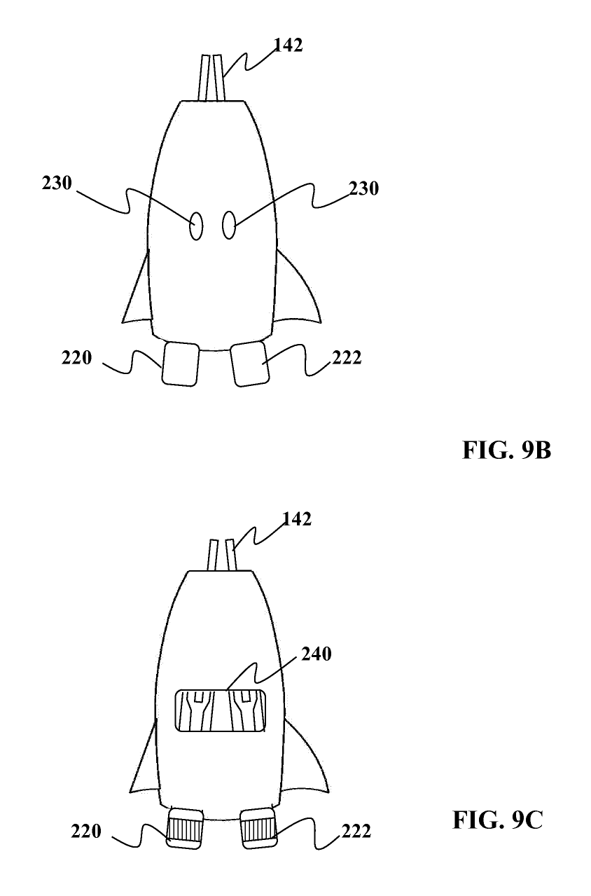

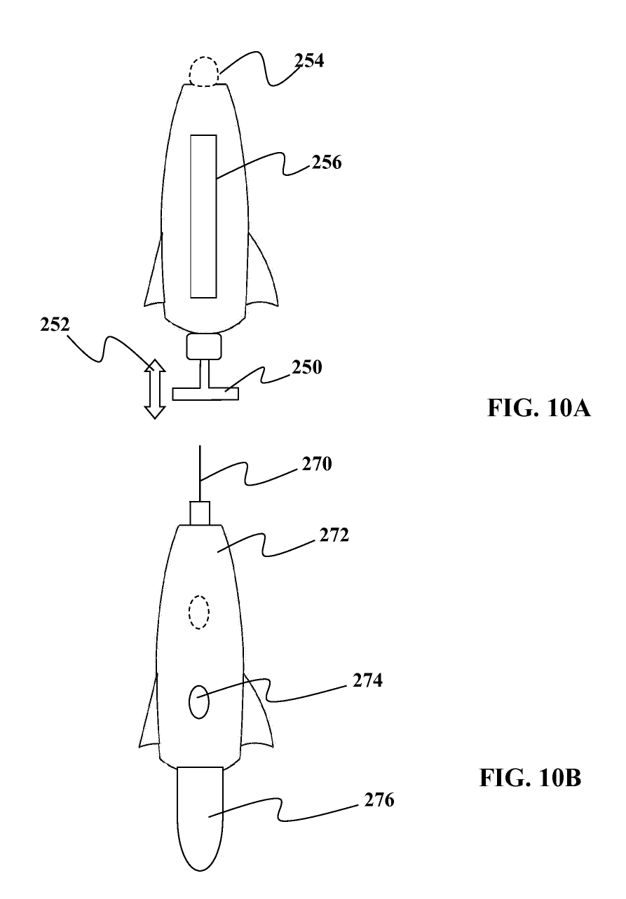

Referring now to FIGS. 9A to 9C, still further embodiments of a novelty exterior sample collection device will now be described. FIG. 9A shows an embodiment wherein two or more sample collection devices are mounted with an exterior shell 200. In one embodiment, this exterior shell comprises a novelty exterior. Optionally, other embodiments may have not a shell decorated or otherwise configured to be a novelty exterior.

FIG. 9A shows that the exterior shell 200 may contain at least two sample collection devices 132 therein. As see in FIG. 9A, the mounting of the collection units 132 within the exterior shell 200 can orient the distal tips of the collection channels 142 to angle towards one another, to decrease the distance between the ends, relative their positions when the collection units 132 as oriented parallel to one another. Optionally, some embodiments can have the collection units 132 in other orientations.

After a desired amount of sample has been collected, the sample collection devices 132 can be removed in the directions indicated by arrows 210 and 212. In one embodiment, this may involve removal of the entire sample collection device 132 from the exterior shell. Optionally, some embodiments may be configured to allow the cap portion 214 to remain in the exterior shell 200 while the other portions of the sample collection devices 132 are removed from the shell. Once removed, a cap 220 or 222 can be removed from a proximal end of the sample collection device 132 and then secured to the open distal end of the sample collection device 132. In this manner, the sample therein can be secured for transport, storage, and/or further processing. FIG. 9B also shows that some embodiments of cap 220 or 222 may have a smooth exterior without texturing.

Referring now to FIGS. 9B and 9C, still other embodiments herein show how windows or other imaging openings are position to allow for determination of sample collection progress and/or success. FIG. 9B shows and embodiment with multiple small openings, windows, or indicators 230 to show the status of sample collection devices 132 therein. Some embodiments may have a transparent covering as part of the indicator 230. Some embodiments may have light emitting or other indicators that can emit light to show status of successful collection and/or collection progress. By way of non-limiting example, the indicators may be used to show one color of light such as green when collection has reached a minimum threshold fill level. Optionally, it may show red if the sample collection device 132 is removed before a minimum threshold fill level is reached. Each indicator can correspond to the status of the corresponding sample collection device 132 inside the exterior shell.

FIG. 9C shows a still further embodiment wherein a single opening, window, or indicator 240 is configured to show the status of sample collection devices 132 inside the exterior shell. Some embodiments may have a transparent covering as part of the indicator 240. Some embodiments may have light emitting or other indicators that can emit light to show status of successful collection and/or collection progress. By way of non-limiting example, the indicators may be used to show one color of light such as green when collection has reached a minimum threshold fill level. Some may have half or some portion show one color, indicating status of a corresponding sample collection device, not all of the units.

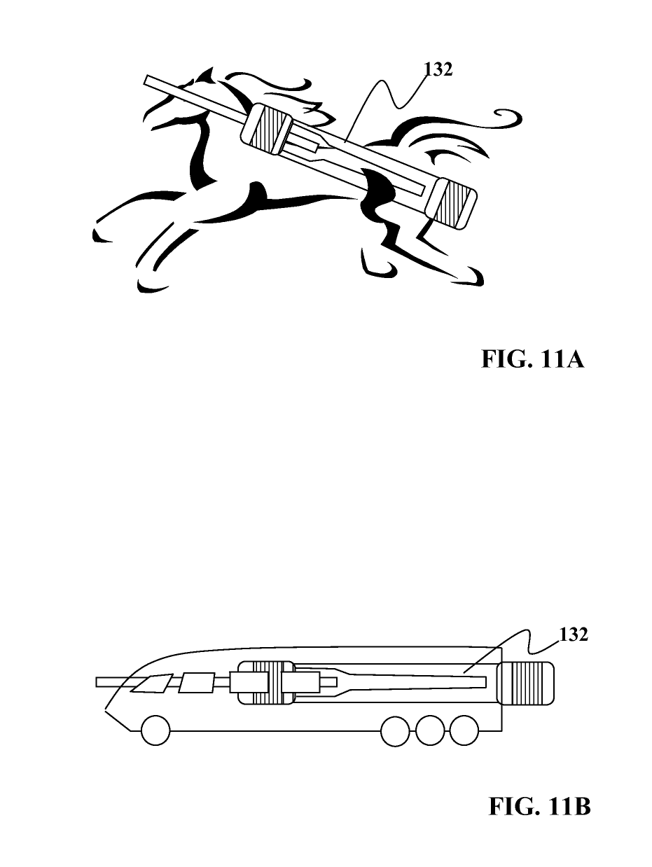

Referring now to FIGS. 10A and 10B, still further embodiments will now be described. FIG. 10A shows an embodiment with a plunger 250 to assist in the draw of sample into the sample collection device within the exterior shell. In this non-limiting example, the plunger 250 may be moved as indicated by arrow 252. Optionally, a container which may or may not have an interior under sub-atmospheric conditions, such as but not limited to full or partial vacuum, can be provided in place of or with the plunger 250. Optionally, the distal end of the exterior shell may include a scoop or other tissue interface 254 (shown in phantom) to improve sample collection. Optionally, the distal end may have a concave or a convex shaped tissue interface surface to engage a tissue or sample for collection. Some embodiments may also include a view window, opening, or indicator 256 to show the status of sample collection device(s) therein. Some embodiments may have a transparent covering as part of the indicator 256. Some embodiments may have light emitting or other indicators that can emit light to show status of successful collection and/or collection progress. By way of non-limiting example, the indicators may be used to show one color of light such as green when collection has reached a minimum threshold fill level. Optionally, it may show red if the sample collection device is removed before a minimum threshold fill level is reached.

FIG. 10B shows a still further embodiment wherein a needle 270 is provided for use in a sample collection procedure, such as but not limited to venipuncture. In one embodiment, a novelty exterior 272 is provided over the sample collection. The novelty feature can be any of the novelty exteriors described herein. Optionally, a container 274 which may or may not have an interior under sub-atmospheric conditions, such as but not limited to full or partial vacuum, can be provided. Optionally, a container with a plunger 250 can be used to draw sample from the needle.

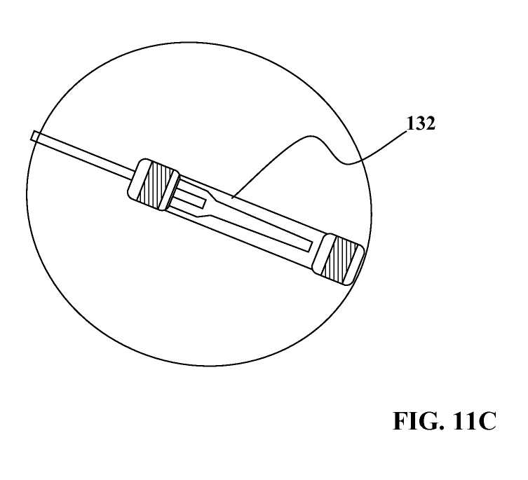

Referring now to FIGS. 11A to 11C, additional embodiments of novelty exteriors are shown. Although FIGS. 11A to 11C show the sample collection device in the form of unit 132, it should be understood that any of the other embodiments of the sample collection device described herein may be configured for use with the novelty exteriors described.

For example, FIG. 11A shows a sample collection device 132 mounted within a toy unicorn, horse, or other animal-style novelty exterior. In one embodiment, a distal end of the sample collection device 132 may be positioned to correspond to a protrusion on the novelty exterior such as but not limited to a horn on the unicorn, spout of an elephant, a horn of rhinoceros, or other similar protruding feature. Optionally, some may locate the collection end of the sample collection device 132, which in some embodiments is also the distal end, at a mouth, snout, or other natural inlet on an animal represented by the novelty exterior. It should be understood that in most embodiments, the sample collection device 132 is removable from the novelty exterior.

FIG. 11B shows an embodiment wherein the novelty exterior may be representative or suggestive of a transportation vehicle. In the embodiment of FIG. 11B, a locomotive or similar vehicle is shown. Optionally, other embodiments may use other train vehicles, ships, airplanes, rockets, missiles, space ships, starships, submarines, or other vehicles or vessels as the novelty exterior.

FIG. 11C shows an embodiment wherein the novelty exterior may be representative or suggestive of a geometric shape. In the embodiment of FIG. 11C, a geometric shape such as but not limited to a disc or sphere is shown. Optionally, other embodiments may use other geometric shape such as but not limited to a square, triangle, pentagon, hexagon, polygon, or the like as the novelty exterior. In some embodiments, there may be decorative drawings on these shapes such as but not limited to smiley face(s), holiday ball decorative pattern, clam shell, bulls-eye target, logo, advertisement, or the like. In one non-limiting example, the information, drawing or the like on the geometric shape may be used to incorporate the geometric shape into the design. Optionally, the geometric shape merely provides a work surface and is not integrated into the design drawn, printed or otherwise provided on the shape.

Some embodiments may include the sample collection device pre-mounted into the novelty exterior such that there is no assembly by the technician of the novelty exterior and the sample collection device. Optionally, some embodiments may have the novelty exterior separate from the sample collection device, such as but not limited to sterility purposes, and that the technician may assemble them at the time of or near use, while maintaining sterility of the collection port(s) which may or may not covered by a protective seal.

By way of non-limiting example and applicable to many of the embodiments herein, a) the collection channel may connect directly to b) a sample container by way of relative motion between one or both of those elements. One or both of the elements may have a novelty exterior. These elements may have intermediate elements connecting them together.

By way of non-limiting example, one or more adapter channels may be discrete elements not initially in direct fluid communication with either the collection channel or the sample containers. Herein the collection channel may connect to the container by way of relative motion between one or more of the collection channel, the adapter channel(s), or the container (sequentially or simultaneously) to create a fluid pathway from the collection channels through the one or more adapter channels into the containers.

By way of non-limiting example, one or more adapter channels may be elements initially in contact with the containers. The adapter channels may not be directly in communication with the interior or the containers. Herein the sample collection channel(s) may connect to the sample container(s) by way of relative motion between one or more of those elements (sequentially or simultaneously) to create a fluid pathway from the collection channels through the one or more adapter channels into the containers. Some embodiments may have a septum, sleeve, sleeve with vent, or cover over the end of the collection channel which will be engaged by the adapter channel. The engagement of the various elements may also move the adapter channel into the interior of the container, as initially, the adapter channel may not be in fluid communication with the interior. In some embodiments, the adapter channel may be a needle. Optionally, some embodiments herein may have more than adapter channel and some embodiments may use adapter channels with pointed ends on both ends of the channel. There may be variations and alternatives to the embodiments described herein and that no single embodiment should be construed to encompass the entire invention.

By way of example and not limitation, embodiments may heat a finger or other target site to improve blood flow and thus blood yield from a finger-stick. For example, the table may have thermal control areas to increase blood flow to the target area and thus increase the speed with which sufficient blood or other bodily fluid can be drawn from the subject. The heating is used to bring the target tissue to between about 40° C. to about 50° C. In embodiments, the heating brings target tissue to within a temperature range of between about 40° C. to about 44° C. In embodiments, the heating brings target tissue to within a temperature range of between about 41° C. to about 43° C. In embodiments, the heating brings target tissue to a temperature of about 42° C. In embodiments, the heating brings target tissue to within a temperature range of between about 44° C. to about 47° C. In one embodiment, the temperature is sufficient to increase blood flow to yield 120 uL of sample. In one embodiment, the temperature is sufficient to increase blood flow to yield 130 uL of sample. In one embodiment, the temperature is sufficient to increase blood flow to yield 140 uL of sample. In one embodiment, the temperature is sufficient to increase blood flow to yield 150 uL of sample. Optionally, the thermal controlled site is a shaped surface is contoured to match that of the target site on a patient.

In embodiments, the thermal controlled site comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

In embodiments, a warming table is configured to place the hand or other body part from which a blood sample is to be collected at an optimal height and orientation for facilitating the flow of gravity in the finger as blood droplets form. A warming table may include a rest, such as an arm rest, or hand rest, or other rest which provides an additional surface for contacting a subject; such a surface may make positioning of a hand, or arm, or foot, or leg, other body part more comfortable for the subject, more convenient for the sample collection technician, or both. Such a rest may be configured for placement in more than one position or orientation. Such a rest may be retractable or otherwise configured to be placed or stored out of the way of a subject or sample collection technician when not needed. A warming table may be designed to accommodate the seating or placement of a sample collection technician near to a subject. A warming table, or surface thereof, may be adjustable, or may include an adjustable surface, which may be placed or oriented for the comfort and convenience of a subject, a sample collection technician, or both. For example, a warming table may include a sliding surface which may be extended or retracted as needed to accommodate a subject or a sample collection technician; may include a tiltable surface which may be oriented as needed to accommodate a subject or a sample collection technician; may include a surface which may be raised or lowered as needed to accommodate a subject or a sample collection technician; or be otherwise adjustable.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, it should be understood that although some embodiments may refer to the warmer as a fingertip warmer, some embodiments can heat other portions of the finger or have an opening at a distal end that allows some portion of the fingertip to extend outside of the warmer. In this manner, warmers are not limited to only novelty exterior, sample collection devices. It should also be understood that although most of the embodiments herein discuss heating the target tissue, it should also be understood that the system can also be used for other thermal conditioning such as but not limited to cooling. Some embodiments may have a cooling object in one pouch and a heating element in another pouch. Some embodiments may use variations of heating and cooling, warm and warmer zones, cool and cooler zones, or other configurations to tailor custom thermal profiles at or near a target tissue. It should also be understood that in some embodiments, thermal element is sized to be larger than the pouch so that at least a portion extends beyond the pouch. This can be useful for ease of removal and/or for increasing the amount of area being treated.

Optionally, although many embodiments herein are described as formed parts with three-dimensional form, some other embodiments may use sticker or planar type covering to be placed over the sample collection device to simulate a fish or other novelty exterior as otherwise described herein. Some embodiments may use an envelope, sleeve, or pull-over type configuration wherein such a pull-over type cover can be transported as in substantially flat configuration and then “opened” along at least one end to allow for insertion of the sample collection device therein. It should be understood that this and optionally at least some or all of the other embodiments herein may be stored in individual-style or batch-style sterile containers, sterile bags, sterile container with burstable wrapping, or other sterile container. Optionally, some embodiments may have stickers, seals, or the like to cover an opening(s) on the novelty cover. In some embodiments, the seals may provide hermetic seals.

Additionally, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a size range of about 1 nm to about 200 nm should be interpreted to include not only the explicitly recited limits of about 1 nm and about 200 nm, but also to include individual sizes such as 2 nm, 3 nm, 4 nm, and sub-ranges such as 10 nm to 50 nm, 20 nm to 100 nm, etc . . . .

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. The following applications are fully incorporated herein by reference for all purposes: U.S. Provisional Application Ser. No. 61/902,777 filed Nov. 11, 2013.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Any feature, whether preferred or not, may be combined with any other feature, whether preferred or not. The appended claims are not to be interpreted as including means-plus-function limitations, unless such a limitation is explicitly recited in a given claim using the phrase “means for.” It should be understood that as used in the description herein and throughout the claims that follow, the meaning of “a,” “an,” and “the” includes plural reference unless the context clearly dictates otherwise. For example, a reference to “an assay” may refer to a single assay or multiple assays. Also, as used in the description herein and throughout the claims that follow, the meaning of “in” includes “in” and “on” unless the context clearly dictates otherwise. Finally, as used in the description herein and throughout the claims that follow, the meaning of “or” includes both the conjunctive and disjunctive unless the context expressly dictates otherwise. Thus, the term “or” includes “and/or” unless the context expressly dictates otherwise.

This document contains material subject to copyright protection. The copyright owner (Applicant herein) has no objection to facsimile reproduction of the patent documents and disclosures, as they appear in the US Patent and Trademark Office patent file or records, but otherwise reserves all copyright rights whatsoever. The following notice shall apply: Copyright 2013-2014 Theranos, Inc.

What is claimed is:

1. A method for obtaining a sample from a subject, said subject having a body part, comprising:
placing a novelty exterior over a sample collection device; and
obtaining a sample from said body part of said subject using said sample collection device with the novelty exterior.

2. The method of claim 1 further comprising creating a wound site on said body part for sample to gather on a surface of the body part for sample collection.

3. The method of claim 1 wherein the sample collection device comprise a capillary channel in fluid communication with a sample collection container.

4. The method of claim 1 further comprising distracting the subject with the novelty exterior while obtaining the sample.

5. The method of claim 1 wherein the novelty exterior covers a portion but not all of the sample collection device.

6. The method of claim 1 further comprising removing a sterility barrier from at least one inlet on the sample collection device.

7. The method of claim 1 further comprising removing a sterility barrier covering at least one inlet and at least a pre-determined sterile zone on the sample collection device around said inlet.

8. The method of claim 1 further comprising, after collecting the sample, removing only a portion of the sample collection device containing the sample while leaving behind a capillary collection portion.

9. The method of claim 1 further comprising, after collecting the sample, removing the novelty exterior from the sample collection device and giving it to the subject as a souvenir.

10. The method of claim 1 further comprising, after collecting the sample, removing the novelty exterior from the sample collection device, sterilizing all or some portion of the novelty exterior, and giving it to the subject as a souvenir.

11. The method of claim 1 further comprising preparing a target location on said body part by warming of at least the target site on said body part with at least one warming device selected from the group consisting of: warming table; a warming plate; a fingertip warmer; an air-warmer; furniture for seating comprising a warming plate or other heating element; and a combination thereof.

12. An assembly comprising:
a sample collection device; and
a novelty exterior coupled to the sample collection device.

13. The assembly of claim 12 wherein the novelty exterior comprises an interior that defines a cavity for receiving the sample collection device.

14. The assembly of claim 12 wherein the novelty exterior is configured to represent a fish-like structure to the subject.

15. The assembly of claim 12 wherein the novelty exterior is configured to represent a train-like structure to the subject.

16. A method for obtaining a sample from a subject, said subject having a body part, comprising:
obtaining a sample from said body part of said subject using a sample collection device with a novelty exterior.
